(19) United States
(12) Patent Application Publication
(10) Pub. No.: US 2022/0175671 A1
(43) Pub. Date: Jun. 9, 2022

(54) ROCKET-SHAPED RECTAL SUPPOSITORY

(71) Applicant: Gail Gentile, North Palm Beach, FL (US)

(72) Inventor: Gail Gentile, North Palm Beach, FL (US)

(21) Appl. No.: 17/116,202

(22) Filed: Dec. 9, 2020

(51) Int. Cl. A61K 9/02

(52) U.S. Cl. CPC A61K 9/025

(57) ABSTRACT

A rocket-shaped rectal suppository configured to retain medication at the site of the hemorrhoids and resists traveling too far inside the rectum. The present rocket-shaped rectal suppository is configured with a small tip for easy insertion and a wider base to prevent it from falling out or getting lost in your rectum. The rocket-shape of the present rectal suppository also provides simultaneous treatment of both internal and external hemorrhoids.

### BACKGROUND

### 1. Field

One or more embodiments of the present invention relate to a rocket-shaped rectal suppository for the treatment of hemorrhoids having an improved shape. In particular, the improved shape of the present rectal suppository retains the medication at the site of the hemorrhoids and prevents the device from slipping out of the rectum. Additionally, the present rocket-shaped rectal suppository provides for the static delivery of extended release medications into the rectum.

### 2. Description of the Related Art

Hemorrhoids are characterized by swollen veins in the lower rectum. Typically, hemorrhoids are caused by strain during bowel movements, obesity or pregnancy. Treatment for hemorrhoids may include home treatments such as consumption of a high-fiber diet, application of an over-the-counter hemorrhoid cream or suppository containing hydrocortisone, or a pad containing a numbing agent. In certain circumstances, a doctor may recommend surgical removal of the hemorrhoid. However, for mild discomfort caused by hemorrhoids, use of a rectal suppository is quite often effective at relieving pain and itching.

Hemorrhoids can occur internally and externally. Internal hemorrhoids are typically far enough inside the rectum that they are not seen. However, symptoms of internal hemorrhoids can include bleeding and prolapse. External hemorrhoids are the most common and often occur in the lower rectum under the skin around the anus. Due to the irritation to the skin caused by external hemorrhoids, they can be quite painful. The excess skin resulting from the irritation can result in itching and inflammation.

Common rectal suppositories for the treatment of hemorrhoids are pill shaped and are administered by insertion into the rectum. The pill shape of rectal suppositories permits relative ease of insertion, but also allows for movement into the rectum away from the hemorrhoid or for slippage out of the rectum entirely. In order for the pill shaped rectal suppository to provide effective treatment of hemorrhoids, the suppository must be inserted just inside the rectum. Unfortunately, due to the pill shape, the common rectal suppository is prone to slippage, decreasing its efficacy. Another type of suppository is molded in the shape of a bullet. However, these bullet-shaped suppositories suffer from many of the same disadvantages, such as slippage and inability to retain medication at the hemorrhoid site.

Some rectal suppositories have been developed that attempt to overcome the slippage resulting from the traditional pill shape. For example, U.S. Pat. Nos. 3,894,539; 4,263,914; 4,563,182; and 4,583,542 disclose devices for insertion into the rectum for the treatment of hemorrhoids. However, these prior attempts either require specific formulations inserted in the device that liquify in the rectum after insertion, or require medication be applied to the rectum prior to insertion of the device. Additionally, these prior attempts are not configured to treat external hemorrhoids.

Therefore, what is needed is a rocket-shaped rectal suppository that avoids the slippage attributed to conventional bullet-shaped suppositories and is configured for the application of traditional and novel formulations for the treatment of internal and external hemorrhoids.

### SUMMARY

In view of what is known in the art, the inventor has developed a rocket-shaped rectal suppository that overcomes the aforementioned disadvantages. The present rocket-shaped rectal suppository is configured to retain medication at the site of the hemorrhoids and does not travel too far inside the rectum. The present rocket-shaped rectal suppository is configured with a small tip for easy insertion and a wider base to prevent it from falling out or getting lost in your rectum. The rocket-shape of the present rectal suppository also provides treatment of both internal and external hemorrhoids.

In one embodiment, the rocket-shaped rectal suppository is a device with a small tip on a top portion and a wider base on a bottom portion, and where the device has a longitudinal slit-like cavity along its vertical length. The advantage of the wider base on the bottom portion of the rocket-shaped rectal suppository is the delivery of medication directly onto external hemorrhoids. Upon insertion into the rectum, the rocket-shaped suppository slowly dissolves inside the body and delivers the medication to treat internal and external hemorrhoids simultaneously.

### BRIEF DESCRIPTION OF THE DRAWINGS

These and/or other aspects will become apparent and more readily appreciated from the following description of the embodiments, taken in conjunction with the accompanying drawings in which:

FIG. 1 is a view of the rectal suppository lying on its side.

FIG. 2 is a front view of the rectal suppository upright on its base.

### DETAILED DESCRIPTION

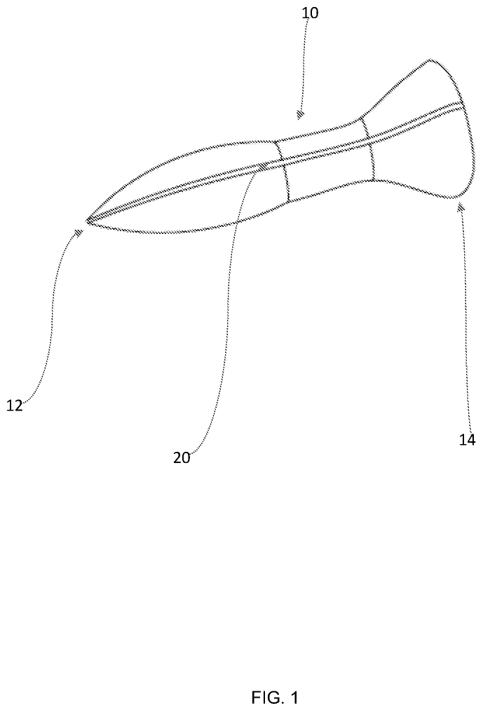

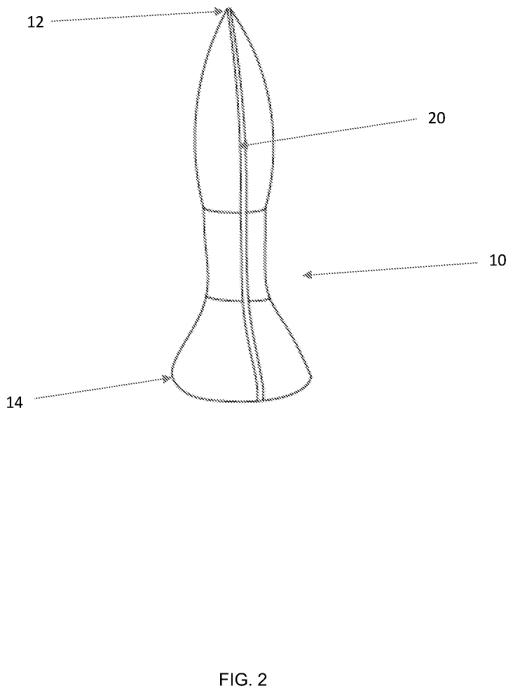

In one embodiment as shown in FIGS. 1 and 2, the rocket-shaped rectal suppository comprises a body member 10 with a small rocket tip 12 and a wider base 14 on the bottom portion, wherein the body member comprises a slit 20 along its vertical length from the small rocket tip to the wider base bottom portion. The slit 20 is configured as a channel to assist the user in releasing gas from the digestive system through the anus (flatulence).

The rocket-shaped rectal suppository comprises a pharmaceutical formulation molded into a rocket-shape as depicted in FIGS. 1 and 2. The process for preparing the mold may be any suitable process known to those skilled in the pharmaceutical compounding art.

The shape of the wider base 14 of the rectal suppository allows for hemorrhoid medication to be retained against the lower rectum under the skin around the anus for the treatment of external hemorrhoids, while the rocket-shape of the top portion of the rectal suppository retains medication inside the rectum for treatment of internal hemorrhoids. An additional advantage of the wider base 14 is to keep the rocket-shaped suppository in place so that it doesn't move further up into the rectum and away from the hemorrhoid site. Currently marketed bullet-shaped suppositories do not have a base and are prone to movement inside the rectum away from the hemorrhoid site resulting in reduced efficacy.

In an alternative embodiment, the rocket-shaped rectal suppository does not comprise a slit along its vertical length. The rectal suppository in this configuration is a device with a small tip on a top portion and a wider base on a bottom portion in a rocket shape.

The rocket-shaped rectal suppository comprises hemorrhoid treatment medication and thickening agents. The medication and thickening agents are suitably mixed together and are poured into a mold or form. The mixture is allowed to harden to a consistency sufficient to permit insertion into the rectum and removed from the mold. In one embodiment, the rocket-shaped rectal suppository is formulated to dissolve slowly to deliver medication to internal and external hemorrhoid sites.

The hemorrhoid medication may be any suitable formulation or composition including, but not limited to, custom blends of at least one active ingredient present in a therapeutically effective amount; such as analgesic and anti-inflammatory ingredients that are FDA-approved to treat hemorrhoids, analgesics, and pain relievers.

The thickening agents of the present rocket-shaped rectal suppository may be any suitable thickening agent or thickener, which, when added to a mixture, increase its viscosity without substantially modifying its other properties.

In one further embodiment, a process for preparing the rocket-shaped rectal suppository includes preparing a formulation or mixture comprising hemorrhoidal medication and thickening agents; pouring the formulation into a mold; hardening the mixture in the mold to a consistency sufficient to permit insertion into the rectum; and removing the hardened rocket-shaped rectal suppository from the mold.

The method for treating internal and external hemorrhoids simultaneously comprises inserting the small rocket tip of the rectal suppository into the rectum until the base of the rectal suppository rests against the outside of the rectum; and allowing the rocket-shaped rectal suppository to slowly dissolve to release medication to the hemorrhoid sites.

The foregoing descriptions of specific embodiments of the present invention have been presented for purposes of illustration and description. They are not intended to be exhaustive or to limit the invention and method of use to the precise forms disclosed. It is understood that many modifications and variations are possible in light of the above teaching. The embodiment was chosen and described in order to best explain the principles of the invention and its practical application, and to thereby enable others skilled in the art to best utilize the invention and various embodiments with various modifications as are suited to the particular use contemplated. It is understood that various omissions or substitutions of equivalents are contemplated as circumstance may suggest or render expedient but is intended to cover the application or implementation without departing from the spirit or scope of the claims of the present invention.

1. A rocket-shaped rectal suppository for the simultaneous treatment of internal and external hemorrhoids wherein the suppository comprises a pharmaceutical formulation molded into a rocket-shape; wherein the rocket-shape comprises a body member with a small rocket tip and a wider base on the bottom portion, and wherein the body member comprises a slit along its vertical length from the small rocket tip to the wider base bottom portion.

2. The suppository of claim 1 wherein the pharmaceutical formulation comprises at least one hemorrhoid medication and at least one thickening agent.

3. A rocket-shaped rectal suppository for the simultaneous treatment of internal and external hemorrhoids wherein the suppository comprises a pharmaceutical formulation molded into a rocket-shape; wherein the rocket-shape comprises a body member with a small rocket tip and a wider base on the bottom portion, and wherein the body member does not comprise a slit along its vertical length from the small rocket tip to the wider base bottom portion.

4. The suppository of claim 3 wherein the pharmaceutical formulation comprises at least one hemorrhoid medication and at least one thickening agent.

5. A method for treating internal and external hemorrhoids simultaneously comprising inserting the small rocket tip of the rectal suppository of claim 1 into the rectum until the base of the rectal suppository rests against the outside of the rectum; and allowing the rocket-shaped rectal suppository to slowly dissolve to release medication to the hemorrhoid sites.

6. A method for treating internal and external hemorrhoids simultaneously comprising inserting the small rocket tip of the rectal suppository of claim 3 into the rectum until the base of the rectal suppository rests against the outside of the rectum; and allowing the rocket-shaped rectal suppository to slowly dissolve to release medication to the hemorrhoid sites.
